(19) United States
(12) Patent Application Publication
(10) Pub. No.: US 2021/0073561 A1
(43) Pub. Date: Mar. 11, 2021

(54) METHOD AND DEVICE FOR DETECTING YAWNING OF A VEHICLE DRIVER

(71) Applicant: FAURECIA INTERIEUR INDUSTRIE, Nanterre (FR)

(72) Inventor: Omar BEN ABDELAZIZ, Tille (FR)

(21) Appl. No.: 17/017,631

(22) Filed: Sep. 10, 2020

(30) Foreign Application Priority Data
Sep. 10, 2019 (FR) FR19 09925

(51) Int. Cl. G06K 9/00; G10L 25/51; A61B 5/024; A61B 5/00

(52) U.S. Cl. CPC G06K 9/00845; G10L 25/51; G06K 9/00248; A61B 2562/029; A61B 5/024; A61B 5/6893; A61B 2560/0252; G06K 9/00375

(57) ABSTRACT

A method for detecting yawning of a driver in a passenger compartment of a vehicle. The method includes: capturing images of at least part of the face of the driver, analyzing the images captured by an image analysis module in order to detect an opening of the driver's mouth, recording sound in the passenger compartment of the vehicle using at least one microphone, detecting a yawn of the driver using a yawn detection module based on the results of the image analysis and the sound recording, an opening of the mouth potentially corresponding to a yawn being considered an actual yawn if it is done in combination with a sound level in the passenger compartment, during the opening of the mouth, below a determined sound threshold.

### TECHNICAL FIELD

The present invention relates to a method for detecting yawning of a driver of a vehicle, in particular an automotive vehicle, for example a light automotive vehicle.

### BACKGROUND

In order to limit the risk of accidents due to fatigue by the driver of a vehicle and the resulting lack of vigilance, it is possible to equip the vehicle with a device for detecting the fatigue state of the driver, also called “fatigue detection device” or “fatigue detector”, able to detect the signs of fatigue of the driver and to alert the driver accordingly, for example by displaying a visual alert signal on the dashboard of the vehicle, emitting a sound alert signal and/or generating vibrations perceptible by the driver, for example in the driver's seat and/or in the steering wheel of the vehicle.

It is desirable for such a device for detecting the fatigue state of the driver to be able to emit the alerts relevantly, so as not to disturb the driver and to prevent the driver from ignoring the alerts due to an excessive number of inappropriate alerts.

### SUMMARY

One aim of the invention is to propose a method for detecting yawning of a vehicle driver that can detect yawning of a driver relevantly.

To that end, the invention proposes a method for detecting yawning of a driver in a passenger compartment of a vehicle, the method comprising: capturing images of at least part of the face of the driver using an image capture device arranged so that each image includes a representation of the mouth of the driver, analyzing the images captured by an image analysis module in order to detect an opening of the driver's mouth, recording sound in the passenger compartment of the vehicle using at least one microphone, detecting a yawn of the driver using a yawn detection module based on the results of the image analysis and the sound recording, an opening of the mouth potentially corresponding to a yawn being considered an actual yawn if it is done in combination with a sound level in the passenger compartment that is below a determined sound threshold during the opening of the mouth.

The detection of the opening of the driver's mouth by image capture and captured image analysis makes it possible to detect yawns of the driver. Taking account of the sound level in the passenger compartment during the opening of the mouth makes it possible to distinguish a yawn from another situation in which the driver opens the mouth, for example to speak, shout or sing.

According to specific embodiments, the detection method comprises one or more of the following optional features, considered individually or according to any technically possible combination(s):

the image capture apparatus is arranged such that each captured image includes the eyes of the driver, the image analysis done by the image analysis module including the detection of the blinking of the eyes, and the yawn detection further being done based on the blinking of the driver's eyes;

the yawn detection is done based on the blinking frequency of the driver's eyes;

the image analysis done by the image analysis module comprises determining the position of the driver's head, the yawn detection further being done based on the position of the driver's head;

it comprises measuring the driver's heart rate, the yawn detection done by the yawn detection module further being done based on the driver's heart rate;

it comprises measuring at least one contextual parameter, the yawn detection being further based on each contextual parameter;

one or several of the following contextual parameters are measured and taken into account: the time, daytime, nighttime, the temperature, the level of humidity in the air, the ambient brightness, the carbon dioxide level (CO₂) in the air inside the passenger compartment;

the image analysis done by the image analysis module comprises detecting the presence of a hand in front of the driver's mouth, and the yawn detection is further done based on the presence or absence of a hand in front of the driver's mouth;

the hand present in front of the driver's mouth after the opening of the driver's mouth is interpreted as a sign of yawning.

The invention also relates to a device for detecting yawns of a vehicle driver, the device comprising an image capture apparatus arranged to take images of at least part of the driver's face including the driver's mouth, and an electronic data processing unit comprising an image analysis module and a yawn detection module configured to implement a detection method as defined above.

### BRIEF DESCRIPTION OF THE DRAWINGS

The invention and its advantages will be better understood upon reading the following description, provided solely as a non-limiting example, and done in reference to the appended drawings, in which:

FIG. 1 is a schematic view of a vehicle equipped with a device for detecting yawns of a driver of the vehicle; and

FIG. 2 is a diagram schematically illustrating a method for detecting yawns of a driver implemented by the detection device of FIG. 1.

### DETAILED DESCRIPTION

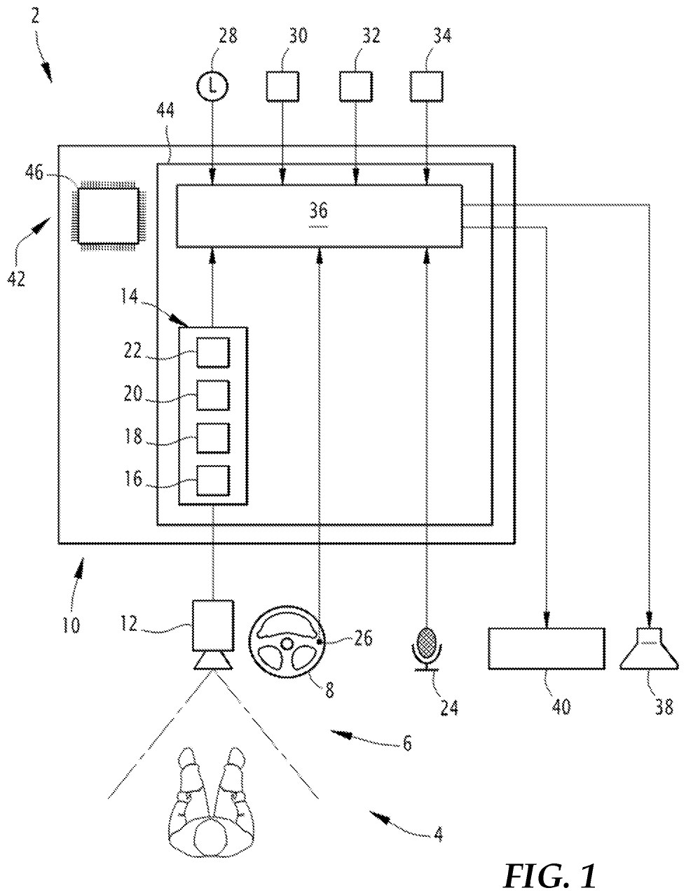

The vehicle 2 illustrated in FIG. 1 is for example an automotive vehicle, in particular a light automotive vehicle, a public transportation vehicle such as a bus or a coach bus, or a heavy vehicle. In a variant, the vehicle is a rail vehicle or an aircraft.

The vehicle 2 has a passenger compartment 4 in which a driver is placed in order to drive the vehicle 2 by means of a driver's post 6 comprising for example a steering wheel 8.

The vehicle 2 is equipped with a device 10 for detecting yawns of a driver, hereinafter called “detection device 10”.

The detection device 10 is configured to detect yawns of the driver installed in the driver's post 6.

The detection device 10 comprises an image capture apparatus 12 arranged to capture images containing a representation of at least part of the face of the driver including the mouth of the driver installed in the driver's post 6.

The image capture apparatus 12 is for example arranged to capture images including a representation of the mouth as well as the eyes of the driver installed in the driver's post 6.

In one exemplary embodiment, the image capture apparatus 12 is arranged to capture images including a representation of the entire face of the driver installed in the driver's post 6.

The image capture apparatus 12 is for example a camera. In a variant, the image capture apparatus 12 is a photographic device.

The detection device 10 is an image analysis module 14 configured to receive and analyze the images captured by the image capture apparatus 12.

The image analysis module 14 comprises a module for detecting opening of the mouth 16 configured to analyze the images so as to detect an opening of the mouth of the driver.

The module for detecting opening of the mouth 16 is configured to detect the depiction of the mouth in the captured images, and to determine whether the mouth is closed or open.

Advantageously, the module for detecting opening of the mouth 16 is configured to determine parameters of the opening of the mouth, for example the amplitude of the opening of the mouth and/or the duration of opening of the mouth.

The module for detecting the opening of the mouth 16 is for example configured to determine the distance between the middle of the lower lip and the middle of the upper lip (vertical opening amplitude of the driver's mouth) and/or the distance between the commissures connecting the lower lip and the upper lip (horizontal opening amplitude of the driver's mouth).

A yawn is generally characterized by a large vertical opening amplitude of the mouth and a large horizontal opening amplitude of the mouth.

Furthermore, a yawn is generally characterized by the maintenance of such an opening of the mouth for a relatively long duration, typically more than 2 seconds.

The image analysis module 14 can be configured to detect other events in the images, for example to refine the detection of the yawns by the driver.

The image analysis module 14 optionally comprises a module for detecting blinking of the eyes 18 configured to detect the depictions of the eyes in the images captured by the image acquisition apparatus 12 and to detect the blinking of the eyes.

The module for detecting blinking of the eyes 18 is for example configured to detect the frequency of the blinking of the driver's eyes.

The blinking of the driver's eyes, and in particular a high frequency of blinking of the driver's eyes, is a sign of fatigue of the driver.

The image analysis module 14 optionally comprises a module for detecting the position of the head 20 configured to detect the position and/or movements of the driver's head.

The position of the driver's head and its movements can reveal a state of fatigue of the driver, for example if the driver's head tends to drop due to fatigue and the driver must regularly lift the head.

The image analysis module 14 optionally comprises a hand detection module 22 configured to detect the presence of a hand in front of the driver's mouth in the captured images.

The hand detection module 22 is configured to detect the hand in the captured images, and to detect the position of the hand relative to the mouth, in particular to detect whether the hand is placed in front of the driver's mouth.

A driver who yawns will tend to place a hand in front of the mouth, whereas he will generally not do so if he is speaking, shouting or singing. In particular, a driver who yawns will tend to place his hand in front of his mouth after the beginning of the yawn. Thus, the detection of the opening of the mouth followed by the positioning of a hand in front of the mouth after the beginning of the opening is a sign of yawning.

The detection device 10 is configured to detect yawns of the driver based on sounds captured in the passenger compartment 4, in particular during a yawn.

The detection device 10 comprises a microphone 24 arranged to capture the sounds in the passenger compartment 4.

Optionally, the detection device 10 is configured to detect the yawn(s) of the driver further based on at least one physiological parameter of the driver.

In one exemplary embodiment, the detection device 10 is configured to detect the yawn(s) of the driver based on the driver's heart rate.

The detection device 10 comprises a heart rate sensor 26 arranged to measure the heart rate of the driver.

The heart rate sensor 26 is for example placed on the steering wheel 8, so as to measure the heart rate on a hand of the driver by which the latter grips the steering wheel 8.

Optionally, the detection device 10 is configured to detect the yawn(s) of the driver based on one or several contextual parameter(s).

Each contextual parameter is for example chosen among the following: the time, the temperature, the level of humidity in the air, the ambient brightness, the carbon dioxide level (CO₂) in the air of the passenger compartment.

The detection device 10 for example comprises a clock 28 making it possible for example to determine the time, a temperature sensor 30, a humidity sensor 32, a brightness sensor 34 and/or a carbon dioxide sensor.

The detection device 10 comprises a yawn detection module 36 configured to detect yawning by the driver based on data supplied by the image analysis module 14 and sounds captured in the passenger compartment by the microphone 24.

The yawn detection module 36 is for example configured to determine the occurrence of a yawn of the driver in case of detection, by the module for detecting the opening of the mouth 16, an opening of the mouth having characteristics corresponding to those of a yawn (e.g., opening amplitude and/or duration), in combination with a sound level in the passenger compartment picked up by the microphone 24 below a determined sound threshold.

In other words, when the yawn detection module 36 identifies, via the module for detecting the opening of the mouth 16, an opening of the mouth that is a potential yawn, the yawn detection module 36 confirms or invalidates that it is an actual yawn based on the sound level measured in the passenger compartment.

Indeed, in case of potential yawn detected in combination with a sound level detected by the microphone 24 greater than the determined sound threshold, it is considered that it is not a yawn. The driver can for example be in the process of opening the mouth to speak, shout or sing.

In case of potential yawn detected in combination with a sound level detected by the microphone 24 below the determined sound threshold, it is considered that the driver is in fact in the process of yawning.

The sound level measured in the passenger compartment via the microphone 24 is therefore used to classify an opening of the mouth potentially corresponding to a yawn, as actually being or not being a yawn.

Optionally, the yawn detection module 36 is configured to detect a yawn of the driver further based on the blinking of the eyes, in particular their frequency, the position of the driver's head, at least one physiological parameter of the driver, for example the heart rate measured by the heart rate sensor 26, and/or at least one contextual parameter, for example the time supplied by the clock 28, the temperature supplied by the temperature sensor 30, the humidity level supplied by the humidity sensor 32 and/or the ambient brightness supplied by the brightness sensor 34.

Taking account of one or several of these additional parameters makes it possible to analyze the behavior of the driver more precisely, and for example to avoid false alarms.

In one exemplary embodiment, the sound threshold is variable and the yawn detection module 36 is configured to determine the sound threshold (used to confirm or invalidate that an opening of the mouth corresponding to a potential yawn is an actual yawn) based on one or several of the aforementioned additional parameters.

In one variant, the sound threshold is invariable. It is therefore predetermined.

The detection device 10 is for example configured to emit a stimulus or several stimuli perceptible by the driver when the detection device 10, more particularly here the yawn detection module 36, determines that the driver is yawning.

The detection device 10 is for example configured to emit a sound stimulus, a visual stimulus and/or a tactile stimulus.

A sound stimulus is for example broadcast by the detection device 10 via at least one electroacoustic transducer 38, for example a speaker of an audio system of the vehicle 2.

A sound stimulus is for example a warning signal or a warning message inviting the driver to take a break.

A visual stimulus is for example broadcast by the detection device 10 via a lighted indicator or a display screen 40 of an infotainment system of the vehicle 2.

A visual stimulus is for example a warning signal or a warning message inviting the driver to take a break.

A tactile stimulus is for example communicated by the detection device 10 via the driver's seat and/or the steering wheel 8, for example in the form of vibrations generated by a vibration generator.

In one exemplary embodiment, the detection device 10 comprises an electronic data processing unit 42 comprising a memory 44 and a processor 46, the image analysis module 14 and/or the yawn detection module 36 being made in the form of software applications and/or computer code instructions recorded in the memory 44 and executable by the processor 46.

In a variant, the image analysis module 14 and/or the yawn detection module 36 is (are) made within the electronic data processing unit 42 in the form of a dedicated electronic circuit or an ASIC (Application-Specific Integrated Circuit) or in the form of a programmable logic circuit, for example an in situ field programmable gate array (FPGA).

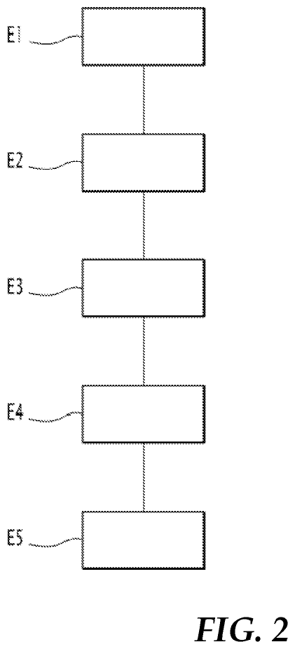

A yawn detection method implemented by the detection device 10 will now be described in reference to FIG. 2.

The detection method comprises an image capture step E1 including a depiction of at least part of the face of the occupant including the driver's mouth, preferably a depiction including the mouth and the eyes, and in particular a depiction including the entire face of the driver.

This step E1 is carried out by the image capture apparatus 12 of the detection device 10.

The detection method comprises a step E2 for detecting the opening of the driver's mouth by analysis of the captured images. The detection step E2 for detecting the opening of the mouth is done by the module for detecting the opening of the mouth 16.

The step for detecting the opening of the mouth for example comprises determining opening parameters of the mouth, for example the shape of the mouth, in particular the distance between the lips and/or the distance between the commissures of the lips and/or the opening duration of the mouth, as previously indicated.

The detection method comprises a step E3 for measuring the sound level in the passenger compartment, for example done using the microphone 24.

The detection method comprises a step E4 for detecting a yawn of the driver based on the detection of the opening of the mouth and the measurement of the sound level, for example done by the yawn detection module 36.

If an opening of the mouth potentially corresponding to a yawn is detected and the sound level is above the determined sound threshold, the yawn detection module 36 determines that the detected opening of the mouth does not correspond to a yawn of the driver.

If an opening of the mouth potentially corresponding to a yawn is detected and the sound level is below the determined sound threshold, the yawn detection module 36 determines that the detected opening of the mouth is a yawn of the driver.

Optionally, if the yawn detection module 36 determines that the driver is yawning, and that he is therefore tired, the yawn detection module 36 can trigger, in a step E5, the generation of a stimulus or of several stimuli to warn the driver of his fatigued state.

In a variant, so as not to disrupt the driver too much, the generation of a stimulus or of several stimuli is triggered when the system detects a certain number of yawn occurrences within a predetermined length of time.

Optionally, the number of yawns and the length of time used to trigger the stimulus or stimuli can be variable and depend on contextual parameters, such as: the time, daytime, nighttime, the temperature, the humidity level in the air, the ambient brightness, the carbon dioxide (CO₂) level in the air inside the passenger compartment.

Optionally, the detection method comprises detecting the blinking of the driver's eyes, the detection of a yawn of the driver accounting for the blinking of the driver's eyes, in particular the frequency of blinking of the driver's eyes.

Optionally, the detection method comprises the detection of the position of the driver's head, the detection of a yawn of the driver accounting for the position of the driver's head.

Optionally, the detection method comprises measuring the heart rate of the driver, the detection of a yawn of the driver further being done based on the heart rate of the driver.

The detection method comprises acquiring and/or measuring at least one contextual parameter (time, temperature, humidity level, ambient brightness, etc.) and the detection of a yawn further being done based on each contextual parameter.

The determined sound threshold used to classify the opening of the mouth as being or not being a yawn is invariable or variable, for example based on one or several of the additional parameters previously indicated.

The opening amplitudes and/or the opening duration of the mouth used to detect the opening of the mouth are invariable or variable, for example based on one or several of the additional parameters previously indicated.

Optionally, the detection method comprises, during the image analysis, detecting the presence of a hand in front of the driver's mouth and detecting yawns of the driver further based on the presence or absence of a hand in front of the driver's mouth.

Owing to the invention, it is possible to detect a yawn of the driver of a vehicle reliably, for example to warn him of his fatigued state suitably, while avoiding false alarms that could cause too many alarms to which the driver would no longer pay attention. The fatigue detection is in fine more effective because it is more precise and better taken into account by the driver, therefore effectively limiting the accident risks related to the lack of attention due to the fatigue of the driver.

1. A method for detecting yawning of a driver in a passenger compartment of a vehicle, the method comprising:
capturing images of at least part of the face of the driver using an image capture device arranged so that each image includes a depiction of the mouth of the driver,
analyzing the image captured by an image analysis module in order to detect an opening of the driver's mouth,
recording sound in the passenger compartment of the vehicle using at least one microphone,
detecting a yawn of the driver using a yawn detection module based on the results of the image analysis and the sound recording, an opening of the mouth potentially corresponding to a yawn being considered an actual yawn if it is done in combination with a sound level in the passenger compartment that is below a determined sound threshold during the opening of the mouth.

2. The detection method according to claim 1, wherein the image capture apparatus is arranged such that each captured image includes the eyes of the driver, the image analysis done by the image analysis module including the detection of the blinking of the eyes, and the yawn detection further being done based on the blinking of the driver's eyes.

3. The detection method according to claim 2, wherein the yawn detection is done based on the blinking frequency of the driver's eyes.

4. The detection method according to claim 1, wherein the image analysis done by the image analysis module comprises determining the position of the driver's head, the yawn detection further being done based on the position of the driver's head.

5. The detection method according to claim 1, comprising measuring the driver's heart rate, the yawn detection done by the yawn detection module further being done based on the driver's heart rate.

6. The detection method according to claim 1, comprising measuring at least one contextual parameter and the yawn detection further based on each contextual parameter.

7. The detection method according to claim 6, wherein one or several of the following contextual parameters are measured and taken into account: the time, daytime, nighttime, the temperature, the level of humidity in the air, the ambient brightness, the carbon dioxide level in the air inside the passenger compartment.

8. The detection method according to claim 1, wherein the image analysis done by the image analysis module comprises detecting the presence of a hand in front of the driver's mouth, and the yawn detection is further done based on the presence or absence of a hand in front of the driver's mouth.

9. The detection method according to claim 8, wherein the hand present in front of the driver's mouth after the opening of the driver's mouth is interpreted as a sign of yawning.

10. A device for detecting yawns of a vehicle driver comprising an image capture apparatus arranged to take images of at least part of the driver's face including the driver's mouth, and an electronic data processing unit comprising an image analysis module and a yawn detection module configured to implement the detection method according to claim 1.
